(19) United States
(12) Patent Application Publication
(10) Pub. No.: US 2011/0004113 A1
(43) Pub. Date: Jan. 6, 2011

(54) METHOD FOR CONTROLLING AND/OR REGULATING A TRAINING AND/OR REHABILITATION UNIT

(76) Inventor: Ulrich Jerichow, Gelnhausen (DE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/741,014

(22) PCT Filed: Oct. 17, 2008
(86) PCT No.: PCT/EP2008/064045

§ 371 Date: Sep. 14, 2010

(30) Foreign Application Priority Data
Nov. 2, 2007 (DE) 10 2007 052 776.6

(51) Int. Cl. A61B 5/083

(52) U.S. Cl. 600/532

(57) ABSTRACT

The invention relates to a method for controlling and/or regulating a training and/or rehabilitation unit, wherein a) a sensor unit is used in the flow of inspiration and expiration air of a person or an animal using the training and/or rehabilitation unit, b) physiological parameters of ventilation and/or gas exchange of the person or the animal are determined using the respiratory gas composition and/or breath volume measured using the sensor unit, c) one or more maximum performance variables are determined on the basis of the determined parameters under submaximal loading, using a regression function and/or by limit loading to the maximum performance capability, and d) a resistance or brake arrangement of the training and/or rehabilitation unit is controlled and/or regulated as a function of at least one of the determined maximum performance variables.

The present invention relates to a method for controlling and/or regulating a training and/or rehabilitation unit as a function of parameters of the respiratory gas composition.

The maximal oxygen uptake (Vo₂ₘₐₓ) is considered the classic parameter for assessing pro-longed physical performance. In addition, the Vo₂ₘₐₓ parameter is used to establish individual training intensities. In order to determine Vo₂ₘₐₓ, the test person/patient must do maximal exhaustive exercise; this involves considerable restrictions with regard to interpretation and use. The Vo₂ₘₐₓ value depends on the weakest link in a chain of physiological processes: the ventilation, the cardiorespiratory capacity, and the local O₂ consumption in the muscles. The test person must be able to do maximal exercise until he/she is completely exhausted. In case of certain diseases (e.g. heart diseases), however, such maximal exercise is definitely out of the question. Alternative parameters for determining physical performance include threshold values lying in the sub-maximal range which are determined using respiratory characteristics and/or the lactate concentrations in the blood. They have long been used in performance diagnostics. On the other hand, the kinetics of the heart rate and of the oxygen uptake allow a more detailed statement in respect of the limiting factors of Vo₂ₘₐₓ.

When searching for an optimum examination approach, the major criteria are minimal exercise intensity and the ability to differentiate. In this way, even patients having a high risk profile can be examined, and an individualized treatment/training strategy is possible on the other hand. This speaks strongly in favour of the kinetics analyses, which in addition have the advantage that the performance protocols used allow direct conclusions as to the threshold values, if required.

The relevant publications available prove clearly that reproducibility is sufficient; comparative studies with healthy test persons have shown that it roughly matches that of the determination of Vo₂ₘₐₓ (1, 2).

The object of the present invention is to provide a method by means of which a person or an animal can, for example, undergo specific fitness or rehabilitation programmes, wherein the training and/or rehabilitation unit used for this purpose can be controlled and/or regulated as a function of parameters of the respiratory gas composition, in particular the Vo₂ₘₐₓ value, of the user, and the person and/or animal need not do maximal exhaustive exercise.

The aforesaid object has been achieved by a method, wherein
a) a sensor unit is arranged in the flow of inspired and expired air of a person or an animal which uses the training and/or rehabilitation unit,
b) the respiratory gas composition and/or the breath volume measured by the sensor unit is/are used to determine physiological parameters of ventilation and/or gas exchange of the person or animal,
c) one or more maximum performance characteristics is/are determined on the basis of the parameters which have been determined
   during sub-maximal exercise, with the aid of a regression function, and/or
   during exhaustive exercise until the performance maximum is reached, and
d) a resistance or brake arrangement of the training and/or rehabilitation unit is controlled and/or regulated as a function of at least one of the parameters which have been determined and/or preferably as a function of at least one of the maximum performance characteristics which have been determined.

The control and/or regulation of the training and/or rehabilitation unit according to the method causes the exercise intensity to be adjusted on the basis of parameters, preferably maximum performance parameters, of the test person or animal which have been determined. All method variants described herein can be used for both humans and animals. The gases oxygen and carbon dioxide are optionally also referred to as O₂ and CO₂, respectively.

According to the inventive method, it is preferred that O₂ uptake (Vo₂), CO₂ output (Vco₂), and/or parameters derived therefrom, namely the respiratory anaerobic threshold (AT), the respiratory quotient (RQ), and/or the oxygen pulse (O_{2Puls}), be determined as gas exchange parameters.

The ventilation parameters which are determined preferably include the tidal volume (VT), the respiratory frequency (fR), and the minute ventilation (VE), and/or the ventilatory equivalent ratio for oxygen (V_{E}/Vo₂) which is derived therefrom.

In a particularly advantageous development of the method, the maximal oxygen uptake (V^{O}₂ₘₐₓ) is determined as the maximum performance characteristic.

The physical performance is usually determined by means of an exercise stress test on a bicycle or treadmill during which exercise intensity is increased step by step. The standard indicator of aerobic capacity is the highest possible oxygen uptake during maximal exercise (Vo₂ₘₐₓ). It indicates the amount of O₂ which is extracted from the inhaled gas in a time unit.

Voₗ is given in l/min; for better comparability, it is expressed relative to the body weight (ml/min/kg). The maximal oxygen uptake is an objective indicator of physical performance; it defines the upper limit of the cardiopulmonary system and is used to assess an individual's state of training and fitness.

These classic methods, however, have the drawback that they require the test person to do maximal exhaustive exercise. For this reason, alternative parameters are increasingly used in performance diagnostics in order to determine physical performance in the sub-maximal exercise range.

In a particularly advantageous development of the method, the maximal oxygen uptake (Vo₂ₘₐₓ) is therefore determined during sub-maximal exercise by means of a regression function. A regression function used for this purpose could, for example, be basically an exponential function according to the equation I:

Vo₂(t)=A_{c}·(1−e^{-t/Tc}), (Equation I),

wherein A_{c} is the asymptotic amplitude and T_{c} is a time constant.

The signal noise in this interrelation between the performance input on the ergometer and the breath-by-breath total oxygen exchange (Vo_{2,t}) can, for example, be minimized by means of a method according to Essfeld (3). In this context, the random or pseudorandom binary sequence (PRBS) method is used. This means the performance of the ergometer changes only between two low exercise levels during a sequence, and the change is made randomly in each case at predefined intervals. Noise is eliminated by calculation, thus enabling small performance amplitudes for the test.

Furthermore, it is advantageous if the resistance or brake arrangement of the training and/or rehabilitation unit is controlled and/or regulated in such a manner that the O₂ uptake (Vo₂) of the person or animal is adjusted to a predefinable partial value of the maximal oxygen uptake (Vo^{2max}).

Preferably, the resistance or brake arrangement of the training and/or rehabilitation unit can be controlled and/or regulated in such a manner that the O₂ uptake (Vo₂) of the person is maintained at a constant value of between 10% and 100%, preferably between 20% and 80%, particularly preferred between 30% and 60%, of the maximal oxygen uptake (Vo₂ₘₐₓ) during exercise. This enables optimum training success. In addition, the training can be adapted to the person's specific form of the day. A training machine could thus be operated at an adequate performance level, so that the person constantly trains with an O₂ uptake (Vo₂) of 40% of his/her Vo₂ₘₐₓ value.

The sensor unit can, for example, be integrated in a breathing mask which is worn by a person or an animal. This arrangement has the particular advantage that the dead volume is extremely small. As an alternative, the sensor unit can be arranged in a headset (a set of headphones and a microphone used for communication) or in a similar means, wherein the only important fact is that the breathing air of the person or animal flows around the sensor unit. A headset according to the present invention is therefore a device which comprises at least a means for holding the sensor unit and a means for affixing the device in the head region of the person or animal. The means for holding the sensor unit must in any case be suited to place the sensor unit in the path of the person's or animal's breathing air. If appropriate, the headset communicates with the other components via a radio link, so that no cable is required.

In addition, an ear clip can be used to measure the oxygen saturation of the blood and/or to measure the pulse of the user, so that comprehensive performance data is recorded and further medical characteristics of the user, for example the heart rate, can be recorded. The measured data which is obtained can advantageously be recorded with the aid of a connected computer, optionally a personal digital assistant (PDA).

The training and/or rehabilitation unit can, for example, be an ergometer, a fitness machine, a cross trainer, a rowing ergometer, a rowing machine, a treadmill, an elliptical trainer, a spin bike, or a bicycle. The resistance and/or brake arrangement of the training and/or rehabilitation unit can, for example, include a pneumatic, hydraulic, mechanical, electromagnetic brake, an eddy-current brake, or a band brake. A training and/or rehabilitation unit can thus, for example, comprise a frame, a means for receiving force, such as pedals, a drive transmission system, a rotating element, and a resistance and/or brake arrangement. In this context, magnetic or electric eddy-current brakes in particular have the advantage that they are easy to control and scarcely susceptible to wear.

The sensor unit can preferably determine the oxygen concentration and/or determine the carbon dioxide concentration with the aid of one or more liquid electrolyte sensor(s).

In an advantageous embodiment, as an alternative to the liquid electrolyte sensor, the sensor unit determines the oxygen concentration with the aid of a heatable electrochemical solid electrolyte sensor, and/or determines the carbon dioxide concentration with the aid of another heatable electrochemical solid electrolyte sensor, and the heating power of heating elements of the sensors is controlled as a function of the breath volume of the person with the aid of a micro-controller in a sensor control unit in order to maintain constant sensor temperatures.

Moreover, it is advantageous if the oxygen sensor contains yttrium-doped zirconium oxide as an electrolyte between two electrodes in order to selectively conduct oxygen ions, and a carrier element, and a heating element, and the carbon dioxide sensor contains an electrolyte made of a super-fast sodium ion conductor, two electrodes, a carrier element, and a heating element (1). The aforesaid super-fast sodium ion conductor, also referred to as NASICON, can be described by means of the formula Na₃₋ₓZr₂(PO₄)₁₊ₓ(SiO₄)₂₋ₓ (2). Sensors of this type have the advantage that they are particularly small and light-weight and can be manufactured at low cost. For example, dimensions of 20×3.5×0.5 mm can be achieved for these sensors (1). Such miniaturized sensors are thus particularly suitable for integration in a breathing mask.

The oxygen concentration in the breathing air is determined in a particularly advantageous manner by measuring the current which, at a constant voltage, flows through the electrolyte of the oxygen sensor from the cathode to the anode, wherein there is a linear relation between the resulting electric current and the oxygen concentration. Furthermore, it is advantageous if the carbon dioxide concentration is determined using a logarithmic relation between the voltage between the electrodes of the carbon dioxide sensor and the carbon dioxide concentration. Furthermore, it is advantageous that the breath volume be determined on the basis of the heating power of the heating elements of the sensors which is controlled by the micro-controller and is required to maintain a constant sensor temperature.

By means of the sensor element, the total flow rate of the breathing air can be determined employing thin-layer anemometry. Moreover, the direction of flow of the breathing gas can be determined either on the basis of the measured oxygen and/or carbon dioxide gradients or of the temperature profile recorded by the sensor. The method according to the invention has the advantage that the volumetric flow rate, the direction of flow, and thus the oxygen and carbon dioxide composition of the inspired air as well as of the expired air can be monitored simultaneously with a breath-by-breath resolution. This means the oxygen and carbon dioxide concentrations can be clearly assigned to the inspired air and the expired air.

The method as a whole or in part can be carried out in a non-invasive manner. The non-invasive variant is less complex and more comfortable for the test person.

Moreover, the method can be carried out using means for two- and three-dimensional visual representation, at least one acoustic output and/or recording means, and means for producing wind, temperature, and/or odour. Moreover, a means for stimulating the sense of touch can be provided. Furthermore, it is advantageous if the components of the training and/or rehabilitation unit, including the resistance and/or brake arrangement which can be controlled and/or regulated, the sensor unit, and the control unit for the sensors, are interconnected by means of a computer system and are controlled and/or read by means of such a computer system. Said computer system can at least consist of a control computer having a user interface.

In an advantageous embodiment variant of the method, a network computer for computing images for the right and left eye is connected to the control computer. The signals generated in this way can be transmitted to a helmet which is worn on the head of the user and is equipped with LCDs for producing a virtual environment (head-mounted display, HMD). As an alternative, the signals which are generated can also be used for a stereo production which serves to produce a three-dimensional representation on a screen. Moreover, it is advantageous if the control computer is connected to one or more input devices having at least six degrees of freedom for position determination and orientation, and said input devices are optionally equipped with one or more buttons. Furthermore, it is advantageous that, for example, isometric, isotonic, and/or elastic input devices be connected to the control computer, wherein said input devices can, for example, be used to detect movement of the eyes, movement of the body, movement of the head, and/or to determine position. In another advantageous development, the input devices can be used to record gestures, facial expressions, and/or speech. This enables a combination of physical and emotional stimuli, and an aroma therapy or high-altitude training in a virtual three-dimensional environment can be carried out.

In another advantageous embodiment variant, the input device is, for example, a head tracker, which can also be affixed to the helmet which is worn on the head of the user and is provided with LCDs for producing the virtual environment (head-mounted display, HMD). Furthermore, it is advantageous that the visual representation unit displays a still image, a moving or non-moving object, a computer graphic, and/or two- and/or three-dimensional moving images or films. Conventional monitors for two-dimensional representation can also be used for this purpose.

In an advantageous development, the visual representation unit can display an image with an angle of view from 0 to 179°, or it can also display an image with an angle of view of 180° or more than 180° for use of the system in the fitness, wellness, or medical fields, wherein moving and/or still real images which have been recorded before by the user can also be displayed.

The acoustic output unit can, for example, play musical instruments, human voices, environmental sounds, such as animal sounds, wind, rain, waterfalls, thunder, and/or sounds of vehicle motors, shots, pumps, explosions, and/or earthwork. It is particularly advantageous if wind, temperature, odour, and/or air humidity can be adapted to the situation which is displayed in the virtual reality.

Moreover, it is advantageous if instructions and/or information can be given to the user of the device by means of a communication unit, and the user can use a communication unit to contact a person which starts the device. In an advantageous further development of the system, blood samples can be taken, thus enabling detailed haemogram analyses, before, during, and/or after use. For example, a cell analysis apparatus which is connected to the computer system, preferably an apparatus for flow cytometry, can be used to exactly determine the composition of the blood cells. In addition, surface markers on the cells can be analysed using specific antibodies, which a preferably coupled to a fluorescent dye.

Moreover, the oxygen content of the expired air could be reduced from 17% to 12%, for example, by increasing the training intensity correspondingly.

Furthermore, the exercise intensity can be individually adapted to maintain a constant ratio (respiratory quotient) of inspired air to expired air by means of the device according to the invention in any training or therapy, regardless of the form of the day or the state of training.

It is further advantageous if a computer program having a program code is used to carry out one or more of the aforesaid method steps according to the invention if the program is executed in a computer. In this context, it is advantageous if the computer program having a program code for carrying out one or more of the aforesaid method steps is stored on a machine-readable carrier if the program is executed in a computer.

The device according to the invention and/or the method according to the invention can, for example, be used by top or competitive athletes to prepare for future competitions by means of high-altitude training units in a virtual environment which is close to reality. In popular and mass sport, on the other hand, training close to reality in oxygen-deficient conditions serves to increase the personal physical performance and individual fitness level. In this way, in particular cost- and time-consuming flights and stays in high mountain regions are no longer required. Moreover, training can be much more efficient since the system is available during 24 hours and can be easily reached in terms of logistics.

In the rehabilitation and wellness fields, the aforesaid system could, for example, combine an aroma therapy with passive high-altitude training and an oxygen therapy in a virtual three-dimensional environment. In such an environment, said combination of relaxation and improvement of the personal physical performance and stimulation of the immune system could be achieved.

In the medical field, the system can be used for an aroma therapy, a high-altitude training and/or an oxygen therapy in a three-dimensional environment, wherein the four senses of sight, touch, smell, and hearing are stimulated. Since the body's defence system is mobilized in this way, it would be conceivable to use the system for people suffering from diseases such as, for example, cancer, allergies, and metabolic disorders.

Moreover, the three-dimensional display technique in particular provides the possibility to use the effect of images and sounds to alleviate specific psychological disorders, such as fears in case of autoimmune diseases.

1. A method for controlling and/or regulating a training and/or rehabilitation unit, wherein
a) a sensor unit is arranged in the flow of inspired and expired air of a person or an animal which uses the training and/or rehabilitation unit,
b) the respiratory gas composition and/or the breath volume measured by the sensor unit is/are used to determine physiological parameters of ventilation and/or gas exchange of the person or animal,
c) one or more maximum performance characteristics is/are determined on the basis of the parameters which have been determined
during sub-maximal exercise, with the aid of a regression function, and/or
during exhaustive exercise until the performance maximum is reached, and
d) a resistance or brake arrangement of the training and/or rehabilitation unit is controlled and/or regulated as a function of at least one of the maximum performance characteristic(s) which have been determined.

2. A method according to claim 1, characterized in that O₂ uptake (Vo₂), CO₂ output (Vco₂), and/or parameters derived therefrom, namely the respiratory anaerobic threshold (AT), the respiratory quotient (RQ), and/or the oxygen pulse (O_{2Puls}), are determined as gas exchange parameters.

3. A method according to claim 1, characterized in that the tidal volume (VT), the respiratory frequency (fR), and the minute ventilation (VE), and/or the ventilatory equivalent ratio for oxygen (V_{E}/Vo₂) which is derived therefrom are determined as ventilation parameters.

4. A method according to claim 1, characterized in that the maximal oxygen uptake (Vo₂ₘₐₓ) is determined as the maximum performance characteristic.

5. A method according to claim 1, characterized in that the resistance or brake arrangement of the training and/or rehabilitation unit is controlled and/or regulated in such a manner that the O₂ uptake (Vo₂) of the person or animal is adjusted to a predefinable partial value of the maximal oxygen uptake (Vo₂ₘₐₓ).

6. A method according to claim 1, characterized in that the resistance or brake arrangement of the training and/or rehabilitation unit is controlled and/or regulated in such a manner that the O₂ uptake (Vo₂) is maintained at a constant value of between 10% and 100% of the maximal oxygen uptake (Vo₂ₘₐₓ) during exercise.

7. A method according to claim 1, characterized in that the sensor unit determines the oxygen concentration and/or determines the carbon dioxide concentration with the aid of one or more liquid electrolyte sensor(s).

8. A method according to claim 1, characterized in that
the sensor unit determines the oxygen concentration with the aid of a heatable electrochemical solid electrolyte sensor, and/or determines the carbon dioxide concentration with the aid of another heatable electrochemical solid electrolyte sensor,
and the heating power of heating elements of the sensors is controlled as a function of the breath volume of the person with the aid of a micro-controller in a sensor control unit in order to maintain constant sensor temperatures.

9. A method according to claim 8, characterized in that the oxygen concentration in the breathing air is determined by measuring the current which, at a constant voltage, flows through the electrolyte of the oxygen sensor from the cathode to the anode, wherein there is a linear relation between the resulting electric current and the oxygen concentration.

10. A method according to claim 1, characterized in that the carbon dioxide concentration is determined using a logarithmic relation between the voltage between the electrodes of the carbon dioxide sensor and the carbon dioxide concentration.

11. A method according to claim 1, characterized in that the breath volume is determined on the basis of the heating power of the heating elements of the sensors which is controlled by the micro-controller and is required to maintain a constant sensor temperature.

12. A method according to claim 1, characterized in that the total flow rate is determined with the aid of the sensor unit employing thin-layer anemometry.

13. A method according to claim 1, characterized in that the direction of flow of the breathing gas is determined either on the basis of the measured oxygen and/or carbon dioxide gradients or of the temperature profile recorded by the sensor.

14. A method according to claim 1, characterized in that the volumetric flow rate, the direction of flow, and thus the oxygen and carbon dioxide composition of the inspired air as well as of the expired air are monitored simultaneously with a breath-by-breath resolution.

15. A computer program having a program code to carry out one or more method steps according to claim 1 if the program is executed in a computer.

16. A computer program having a program code which is stored on a machine-readable carrier for carrying out one or more method steps according to claim 1 if the program is executed in a computer.
